(19) United States
(12) Patent Application Publication
(10) Pub. No.: US 2026/0130963 A1
(43) Pub. Date: May 14, 2026

(54) COMPOSITIONS COMPRISING RED YEAST RICE

(71) Applicant: Meda Pharma S.p.A., Monza (IT)

(72) Inventors: Elena Gelfi, Monza (IT); Manuel Mosconi, Monza (IT); Andrea Zanardi, Monza (IT)

(21) Appl. No.: 18/705,156

(22) PCT Filed: Oct. 31, 2022
(86) PCT No.: PCT/EP2022/080337

§ 371 Date: Apr. 26, 2024

(30) Foreign Application Priority Data
Oct. 29, 2021 (GB) 2115617.9

(51) Int. Cl. A61K 36/899; A61K 9/50; A61K 31/045; A61K 31/122; A61K 31/519; A61K 31/722; A61K 36/062; A61K 36/28; A61K 36/29; A61K 36/53; A61K 36/537; A61K 36/73; A61K 36/752; A61K 36/82; A61K 36/88; A61K 36/9066

(52) U.S. Cl. CPC A61K 36/899; A61K 9/5015; A61K 9/5031; A61K 9/5089; A61K 31/045; A61K 31/122; A61K 31/519; A61K 31/722; A61K 36/062; A61K 36/28; A61K 36/29; A61K 36/53; A61K 36/537; A61K 36/73; A61K 36/752; A61K 36/82; A61K 36/88; A61K 36/9066; A61K 2236/19

(57) ABSTRACT

The invention relates to solid oral compositions, comprising monascus fermented rice coated by lipidic encapsulation technology and a method for preparing the coated monascus fermented rice.

The invention relates to solid oral compositions, based on monascus fermented rice standardized in monacolin K (MK), using specific lipidic encapsulation technology for improving monacolin K solubility and dissolution rate.

### BACKGROUND OF THE INVENTION

Monascus fermented rice, also known as red yeast rice (RYR), has traditionally been used as a natural food colorant and food preservative of meat and fish for centuries. It has recently become a popular dietary supplement because many of its bioactive constituents, including a series of active drug compounds, monacolins (indicated as 3-hydroxy-3-methylglutaryl-coenzyme A reductase inhibitors) have been discovered as being capable of reducing serum cholesterol levels (Lin et al., Appl Microbiol Biotechnol 2008; 77, 965-973).

Among the bioactive compounds found in red yeast rice, monacolins are well known for their pharmacological effects to control hyperlipidemia. Monacolin K is considered the most efficacious compound to lower cholesterol in the plasma. It is structurally identical to lovastatin, and mevinolin (Klimek, Wang, and Ogunkanmi, P&T 2009, 34 No 6, 313-316).

Solubility is one of the most important physicochemical properties in drug release and absorption, playing an integral role in bioavailability, especially for an orally administered drug.

Moreover, for significant bioavailability, the orally administered drug not only depends on its solubility in the gastrointestinal tract but also its permeability across cell membranes. Hence, the drug molecules are required to be presented in a suitable form, to be transported across biological membranes. Also, an essential prerequisite for the absorption of a drug by all mechanisms except endocytosis is that it must be present in aqueous solution. This fact, in turn, depends on the drug's aqueous solubility (absolute or intrinsic solubility) and its dissolution rate (Poovi and Damodharan, Future Journal of Pharmaceutical Sciences 2018; 4, 191-205).

Lovastatin exhibits poor oral bioavailability (5%) because of its poor water solubility (0.4×10⁻³ mg/mL) and short half-life (1-2 hours) (Zhou and Zhou 2015; Drug design, development and therapy 9:5269-5275). The poor bioavailability of an orally administered dose is due to extensive first-pass metabolism. Lovastatin is classified as BCS Class II, with “low solubility/high permeability” therefore, it can be anticipated that the poor oral bioavailability of lovastatin could be due to its limited aqueous solubilization which further poses dissolution limitations. (Qureshi, Chitneni, and Kian Asian Journal of Pharmaceutical Sciences, 2014; 10, 40-56). Due to its structural identity the same is true for monacolin K.

Furthermore, monacolin K may also lack stability in certain oral dosage forms.

Accordingly, it is an object of the present invention to provide monacolin K in a red yeast rice formulation which shows an increased dissolution rate and higher solubility. A further object of the present invention is to provide monacolin K in a red yeast rice formulation which displays improved stability.

### SUMMARY OF THE INVENTION

In a first aspect, the invention provides a composition comprising red yeast rice and a lipid carrier comprising (a) a fatty alcohol and/or a fatty acid; (b) a glyceride; and (c) a polyethylene glycol.

In a second aspect, the invention provides a composition comprising red yeast rice and a lipid carrier comprising (a) a fatty alcohol and/or a fatty acid; (b) a glyceride; and (c) a polyethylene glycol, wherein the composition is obtainable by hot melt granulation.

In a third aspect, the invention provides a process for preparing compositions comprising red yeast rice and a lipid carrier comprising a fatty alcohol and/or a fatty acid, a glyceride, and a polyethylene glycol, wherein the process comprises the following steps:
(i) heating one or more of the lipid carrier components until at least partially melted, preferably completely melted;
(ii) combining the red yeast rice with the one or more melted lipid carrier components until the red yeast rice is incorporated into the molten lipid carrier matrix;
(iii) combining the resulting mixture with any remaining lipid carrier components;
(iv) cooling the resulting mixture until the lipid carrier components have solidified; and
(v) crushing, and optionally sieving, the resulting solid mixture to obtain a granulate.

Surprisingly the applicant has found that compositions comprising red yeast rice and a lipid carrier comprising at least one fatty alcohol or fatty acid, one or more glycerides and one or more polyethylene glycol has improved solubility and bioavailability showing a statistically significant effect. Lipid encapsulated compositions of the invention may also demonstrate improved stability of monacolin K relative to unencapsulated red yeast rice compositions.

The present invention will now be described with reference to the accompanying drawings, in which:

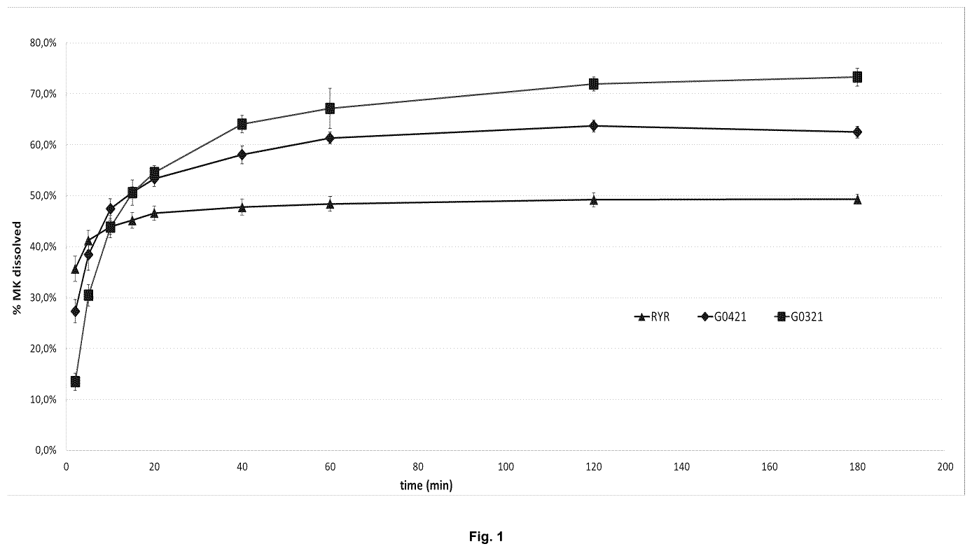

FIG. 1: shows the dissolution rates of monacolin K in encapsulated formulations; and

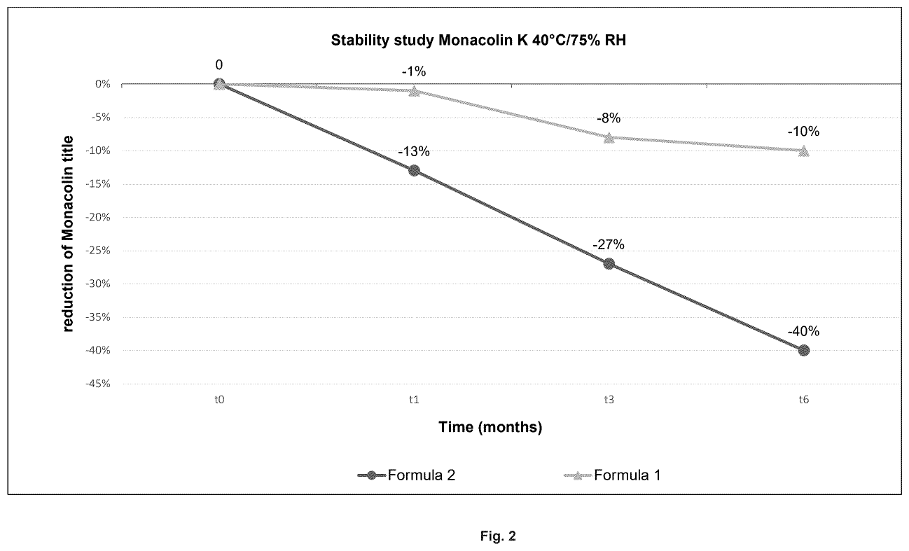

FIG. 2: details a stability study in which the percentage reduction of monacolin K at different time points is shown;

### DEFINITIONS

The proportions of the various components of the combination are defined relative to other components. The wt % (weight percent) of a particular component, based on the other components, is the weight (mass) of the particular component, divided by the weight (mass) of based on weight of the composition, times 100 i.e.

$$wt \% single component \times ( based on weight of the composition Y ) = \frac{wt ( X )}{wt ( Y )} \times 100$$

Monascus fermented rice or red yeast rice is a traditional Chinese preparation of cooked white rice, being fermented with Monascus purpureus for a few days at room temperature, which results in the red colour.

The red yeast rice used in the context of the present invention is standardized in 1.75% (w/w) monacolin K.

### DETAILED DESCRIPTION OF THE INVENTION

The composition includes red yeast rice and a lipid carrier comprising (a) a fatty alcohol and/or a fatty acid; (b) a glyceride; and (c) a polyethylene glycol.

The lipid carrier may encapsulate the red yeast rice, also referred to herein as lipidic encapsulation of the red yeast rice. Alternatively, the lipid carrier may be intimately mixed with the red yeast rice.

Preferably, the invention provides a composition of red yeast rice standardized in monacolin K to 1.75% w/w and a lipid carrier comprising at least one fatty alcohol or fatty acid, a glyceride, and a polyethylene glycol.

Suitable fatty alcohols are cetyl alcohol, stearyl alcohol, palmityl alcohol, myristyl alcohol, arachidyl alcohol, lauryl alcohol, behenyl alcohols, and combinations thereof. A preferred fatty alcohol is cetyl alcohol.

Suitable fatty acids are stearic acid, palmitic acid, myristic acid, arachidic acid, lauric acid, and combinations thereof.

Suitable glycerides are glyceryl monostearate, glyceryl distearate, glyceryl behenate, glyceryl dibehenate, glyceryl tristearate, glyceryl laurate, glyceryl palmitate, glyceryl myristate, glyceryl arachidate and combinations thereof. Preferred glycerides are glyceryl monostearate and glyceryl dibehenate.

Preferred polyethylene glycols (PEG) are those having an average molecular mass of from 400 to 6000 g/mol, in particular of 400 g/mol, 1500 g/mol, 3350 g/mol, 4000 g/mol or 6000 g/mol.

In one embodiment, the lipid carrier may comprise more than one fatty alcohol and/or fatty acid, glyceride and/or polyethylene glycol.

According to one aspect of the invention there is provided a lipidic encapsulation of red yeast rice comprising a fatty alcohol, one or more glycerides, and a polyethylene glycol.

In one embodiment the composition comprises:
a fatty alcohol;
a glyceride; and
PEG 1500.

In another embodiment the composition comprises:
cetyl alcohol;
glyceryl monostearate; and
PEG 1500.

In another embodiment the composition comprises:
cetyl alcohol;
glyceryl dibehenate; and
PEG 1500.

In another embodiment the composition comprises:
cetyl alcohol;
glyceryl monostearate;
glyceryl dibehenate; and
PEG 1500.

In one embodiment the composition comprises:
a fatty alcohol in amount from 0.5 to 13%;
a glyceride in an amount from 79 to 98%; and
PEG 1500 in an amount from 0.5 to 8%.

In another embodiment the composition comprises:
cetyl alcohol in an amount from 0.5 to 13%;
glyceryl monostearate in an amount from 1 to 14%; and
PEG 1500 in an amount from 1 to 8%.

In another embodiment the composition comprises:
cetyl alcohol in an amount from 0.5 to 13%;
glyceryl dibehenate in an amount from 65 to 97%; and
PEG 1500 in an amount from 1 to 8%.

In another embodiment the composition comprises:
cetyl alcohol in an amount from 0.5 to 13%;
glyceryl monostearate in an amount from 1 to 14%;
glyceryl dibehenate in an amount from 65 to 97%; and
PEG 1500 in an amount from 1 to 8%.

In a preferred embodiment, the composition comprises:
cetyl alcohol in an amount from 0.6 to 5.0%;
glyceryl monostearate in an amount from 0.6 to 4.4%; and
PEG 1500 in an amount from 0.6 to 3.1%.

In another preferred embodiment, the composition comprises:
cetyl alcohol in an amount from 0.6 to 5.0%;
glyceryl dibehenate in an amount from 25.0 to 35.7%; and
PEG 1500 in an amount from 0.6 to 3.1%.

In another preferred embodiment, the composition comprises:
cetyl alcohol in an amount from 0.6 to 5.0%;
glyceryl monostearate in an amount from 0.6 to 4.4%;
glyceryl dibehenate in an amount from 25.0 to 35.7%; and
PEG 1500 in an amount from 0.6 to 3.1%.

In another preferred embodiment the composition comprises
cetyl alcohol 0.6%;
glyceryl monostearate 0.6%; and
PEG 1500 0.6%.

In another preferred embodiment the composition comprises
cetyl alcohol 0.6%;
glyceryl dibehenate 35.7%; and
PEG 1500 0.6%.

In another preferred embodiment the composition comprises
cetyl alcohol 0.6%;
glyceryl monostearate 0.6%;
glyceryl dibehenate 35.7%; and
PEG 1500 0.6%.

In another preferred embodiment the composition comprises
cetyl alcohol 5.0%;
glyceryl monostearate 4.4%; and
PEG 1500 3.1%.

In another preferred embodiment the composition comprises
cetyl alcohol 5.0%;
glyceryl dibehenate 25.0%; and
PEG 1500 3.1%.

In another preferred embodiment the composition comprises
cetyl alcohol 5.0%;
glyceryl monostearate 4.4%;
glyceryl dibehenate 25.0%; and
PEG 1500 3.1%.

Suitable encapsulation ratios for component A: fatty alcohols, glycerides, PEG and component B: red yeast rice (standardized in monacolin K) are in the range of wt % ratios of:
0.5 to 1,
0.6 to 1,
1 to 1,
1 to 2.
1 to 3.
1 to 5.

A preferred ratio is 0.6 to 1 wt % of component A to component B.

In a further embodiment, the aforementioned compositions may additionally comprise one or more components selected from Berberis aristata, phytosterols and/or phytostanols, Cynara cardunculus extract, Citrus bergamia extract, Allium sativum, Salvia miltiorrhiza, policosanol, Camellia sinensis extract, Melannurca campana extract, Curcuma longa and curcuminoids, spirulina, chitosan, betaglucan, glucomannan, coenzyme Q10, astaxanthin, folic acid and orthosiphon.

### Preparation

The lipid encapsulated red yeast rice may be prepared by using a hot melt granulation technique.

In one embodiment, the composition including red yeast rice and a lipid carrier comprising (a) a fatty alcohol and/or a fatty acid; (b) a glyceride; and (c) a polyethylene glycol, may be obtainable by hot melt granulation.

The melting or fusion method was first proposed in 1961 to prepare fast release solid dispersion dosage forms. In this method, the physical mixture of a drug and carriers are heated directly until they melt. The melted mixture can be then cooled and solidified rapidly in an ice bath with rigorous stirring. The final solid mass is then crushed, pulverized, and sieved, which can be compressed into tablets with the help of tableting agents.

In one embodiment, the invention provides a process for preparing compositions comprising red yeast rice and a lipid carrier comprising a fatty alcohol and/or a fatty acid, a glyceride, and a polyethylene glycol, wherein the process comprises the following steps:
(i) heating one or more of the lipid carrier components until at least partially melted, preferably completely melted;
(ii) combining the red yeast rice with the one or more melted lipid carrier components until the red yeast rice is incorporated into the molten lipid carrier matrix;
(iii) combining the resulting mixture with any remaining lipid carrier components;
(iv) cooling the resulting mixture until the lipid carrier components have solidified; and
(v) crushing, and optionally sieving, the resulting solid mixture to obtain a granulate.

Preferably, in step (i) of the process, all of the lipid carrier components are heated until they are completely melted.

The method of preparation typically involves heating the one or more of the lipid carrier components until completely melted (65-80° C.). The heating was provided at a constant temperature, between 85° C. and 105° C. until the mass begins to soften.

When the excipients are completely melted, the red yeast rice is added gradually in small amounts allowing the temperature of the molten mass to return to the optimal range described before, until the red yeast rice is completely incorporated into the molten matrix. The mass thus obtained is cooled down, crushed with a mill and calibrated on a sieve of 1 mm, forming a granulate.

The melting point of a binary system is dependent upon its composition, that is, the selection of the carrier and the weight fraction of the drug in the system. An important parameter for the formation of solid dispersion by the hot-melt method is the miscibility of the drug and the carrier in molten form. Another important parameter is the thermostability of drug and carrier. (Savjani, Gaijar, Savjani (2012); ISRN Pharm. 195727).

The encapsulated RYR is mixed with adequate excipients to obtain the required dosage form. The process is suitable for direct mixing and direct compression. The blend thus obtained can be used to prepare the finished dosage form by compression with a rotary tablet-compressing machine equipped with suitable punches, or encapsulation using a capsule filling machine, or dosing into sachets or stickpacks by an adequate packaging machine.

### Uses of the Invention

The invention also provides the use of the compositions disclosed herein for the treatment or prevention of hypercholesterolemia or hyperlipidemia.

The invention also provides the use of the compositions disclosed herein for the manufacture of a medicament for the treatment or prevention of hypercholesterolemia or hyperlipidemia.

Hypercholesterolemia (and hyperlipidemia) is a well-known risk factor for coronary artery, cerebrovascular and peripheral artery diseases. In fact, any reduction of basal cholesterol in plasma levels is correlated to a proportionally reduced incidence of cardiovascular complications (myocardial infarction, stroke, peripheral obstructive arterial disease). The correlation already exists before the first clinical event, relevant for primary prevention, as well as for the cardiovascular events that follow the first clinical vent, relevant for secondary prevention.

### Dosage

The compositions of the invention are useful in the treatment or prevention of hypercholesterolemia, and hyperlipidemia.

The compositions are generally administered to a subject in need of such administration, for example a human or animal, typically a human.

The compositions will typically be administered in amounts that are therapeutically or prophylactically useful.

The compositions may be administered over a prolonged term to maintain beneficial therapeutic effects or may be administered for a short period only.

A typical daily dose of each components of the combination can be in the range from 100 μg to 100 mg per kg of body weight, more typically 5 ng to 25 mg per kg of bodyweight, and more usually 10 ng to 15 mg per kg (e.g. 10 ng to 10 to 20 mg, and more typically 1 μg per kg to 20 mg per kg, for example 1 μg to 10 mg per kg) per kg of bodyweight although higher or lower doses may be administered where required.

The compositions may be administered orally in a range of doses, for example 0.1 to 1000 mg, 1 to 800 mg, 5 to 700 mg, 10 to 500 mg, 25 to 400 mg, or 50 to 350 mg.

Particular examples of daily doses of the composition are 100, 200, 300, 600, 900, 1200, 1500 and 1800 mg.

For a composition of red yeast rice standardized in monacolin K to 1.75% w/w, this dosage range typically corresponds to a daily dose of monacolin K of between 1.75 mg to about 35 mg.

### Formulations

In one embodiment, the lipid encapsulated red yeast rice composition is provided as oral dosage forms. Oral dosage forms include tablets (coated or uncoated), capsules (hard or soft shell), caplets, pills, lozenges, syrups, solutions, powders, granules, elixirs and suspensions, sublingual tablets, wafers or patches such as buccal patches.

Therefore, in one embodiment of the invention, the lipid encapsulated red yeast rice composition is presented in a tablet.

Typically, the tablet includes one or more pharmaceutically acceptable excipient. The pharmaceutically acceptable excipient can be selected from, for example, carriers (e.g. a solid, liquid or semi-solid carrier), adjuvants, diluents, fillers or bulking agents, granulating agents, coating agents, release-controlling agents, binding agents, disintegrants, lubricating agents, preservatives, antioxidants, buffering agents, suspending agents, thickening agents, flavouring agents, sweeteners, taste masking agents, stabilisers or any other excipients conventionally used in pharmaceutical compositions.

Preferably, the composition of the invention is formulated with one or more pharmaceutically acceptable fillers or bulking agents.

Examples of excipients include dibasic calcium phosphate anhydrous, magnesium stearate, silicon dioxide, carboxymethylcellulose, crospovidone, and hydroxypropyl cellulose and maltodextrin.

In one embodiment, the lipid encapsulated red yeast rice composition is provided in capsules.

Typically, the capsule includes one or more pharmaceutically or nutraceutically acceptable excipient. The pharmaceutically or nutraceutically acceptable excipient can be selected from, for example, carriers (e.g. a solid, liquid or semi-solid carrier), adjuvants, diluents, fillers or bulking agents, granulating agents, coating agents, release-controlling agents, binding agents, disintegrants, lubricating agents, preservatives, antioxidants, buffering agents, suspending agents, thickening agents, flavouring agents, sweeteners, taste masking agents, stabilisers or any other excipients conventionally used in pharmaceutical compositions.

Examples of excipients include dibasic calcium phosphate anhydrous, magnesium stearate, silicon dioxide, maltodextrin, carboxymethylcellulose, crospovidone, and hydroxypropyl cellulose.

In one embodiment, the lipid encapsulated red yeast rice composition is provided as granulates. The granulates may be packaged into a sachet or a stick pack.

The granulate may be prepared by dry or wet granulation techniques that are known in the art.

### Formulation Examples

| Lipid encapsulated | | |
|---|---|---|
| RYR composition | % w/w | mg/tab |
| Red yeast rice powder | 62.5 | 200 |
| Cetyl Alcohol | 0.6 | 2 |
| Glyceryl Monostearate | 0.6 | 2 |
| Glyceryl Dibehenate | 35.7 | 114 |
| PEG 1500 | 0.6 | 2 |
| Total | 100.0 | 320 |

| Tablet | % w/w | mg/tab |
|---|---|---|
| Lipid encapsulated RYR composition | 40.0% | 320 |
| Microcrystalline cellulose | 58.0% | 464 |
| Silicon dioxide | 1.0% | 8 |
| Magnesium stearate | 1.0% | 8 |
| Total | 100.0 | 800 |

| Capsule | % w/w | mg/tab |
|---|---|---|
| Lipid encapsulated RYR composition | 49.2 | 320 |
| Maltodextrin | 48.8 | 318 |
| Silicon dioxide | 1.0 | 6 |
| Magnesium stearate | 1.0 | 6 |
| Total | 100.0 | 650 |

| Granulate | % w/w | mg/tab |
|---|---|---|
| Lipid encapsulated RYR composition | 20.6 | 320 |
| D-Mannitol | 77.4 | 1200 |
| Flavour | 0.7 | 11 |
| Sucralose | 0.1 | 1 |
| Silicon dioxide | 1.2 | 18 |
| Total | 100.0 | 1550 |

### Examples

### Dissolution Analysis of Encapsulated Red Yeast Rice

The preparation of the samples for dissolution analysis followed the general procedure as described, using a melting temperature between 70-75° C. After complete incorporation of the red yeast rice into the molten matrix the mass obtained is cooled down to a temperature between 30-42° C.

The aim of the dissolution study was to compare the effect of lipid encapsulation on the dissolution profiles of monacolin K comprising compositions:

| G 0321 | wt % |
|---|---|
| red yeast rice powder | 62.5 |
| glyceryl dibehenate | 35.7 |
| cetyl alcohol | 0.6 |
| glycerol monostearate | 0.6 |
| PEG 1500 | 0.6 |

| G 0421 | wt % |
|---|---|
| red yeast rice powder | 62.5 |
| glyceryl dibehenate | 25.0 |
| cetyl alcohol | 5.0 |
| glycerol monostearate | 4.4 |
| PEG 1500 | 3.1 |

Control:—Red yeast rice powder standardized in monacolin K-“RYR”

### Dissolution Test

1 g of raw material was mixed into 500 ml of dissolution medium, 50 mM phosphate buffer with 0.05% SDS, pH 6.8. The dissolution test was performed using USP Dissolution Apparatus 2 (Paddle. 37° C.±0.5° C.) containing 6 vessels. The paddle rotational speed was 50 rpm. At each time point (t=2 min, 5 min, 10 min, 15 min, 20 min, 40 min, 60 min, 120 min and 180 min), about 3 ml of solution was sampled and filtered on RC 0.2 μm. The filtered samples were analyzed by means of HPLC-UV (Internal method 0028 rev 03) to determine the dissolved amount of monacolin K, at each time point. For all materials, the relative amount of dissolved monacolin K (MK) was calculated as follows:

$$\% dissolved M K = \frac{mg dissolved M K}{mg M K in 1 g of raw material}$$

To approximate the derivative of the curve % dissolved MK vs time at a chosen time point ti, the variation rate at ti of % dissolved MK was calculated as follows:

$$VR ( {t}_{i} ) = \frac{\% dissolved M K ( t i + 1 ) - \% dissolved M K ( t i - 1 )}{t i - 1 - t i - 1}$$

The statistical evaluation was to determine whether the differences in variation rates (at time points 5, 10, 15, 20, 40, 6, 120 and 180 min) between granulated and classical RYR are statistically relevant. Oneway ANOVA was used to test the equality of the means of variation rate (“VR”) in samples RYR, G0321 and G0421. Each sample contained a set of 6 data points. Statistical analyses were performed with Minitab Software.

### Results

The one-way ANOVA tests performed on samples RYR, G0321 and G0421 at different times, supported a statistically significant difference (p<0.05) between the dissolution curves of lipid encapsulated compositions G0421 and G0321 vs RYR (not lipid encapsulated Red Yeast Rice). Both G0321 and G0421, when compared to RYR, showed a significant superior dissolution rate.

TABLE 1
% of Monacolin K dissolved at different time

| | % monacolin K dissolved | | |
|---|---|---|---|
| time/min | G0321 | G0421 | RYR |
| 2 | 13.5 | 27.3 | 35.6 |
| 5 | 30.4 | 38.4 | 41.2 |
| 10 | 43.8 | 47.5 | 43.9 |
| 15 | 50.6 | 50.7 | 45.2 |
| 20 | 54.5 | 53.3 | 46.6 |
| 40 | 64.0 | 58.0 | 47.7 |
| 60 | 67.1 | 61.3 | 48.4 |
| 120 | 71.9 | 63.6 | 49.2 |
| 180 | 73.2 | 62.4 | 49.3 |

### Stability Analysis of Encapsulated Red Yeast Rice

The aim of the stability study was to compare the effect of lipid encapsulation on the dissolution profiles of monacolin K comprising compositions.

For this study the following formulations have been prepared:

Formula 1 - with lipid encapsulated red yeast rice:

| | | |
|---|---|---|
| Microcrystalline cellulose | 157.92 | mg |
| Magnesium stearate | 8.00 | mg |
| Silicum dioxide | 4.00 | mg |
| Folic Acid | 0.26 | mg |
| Encapsulated Red Yeast Rice G 0321 (1.2%) | 243.00 | mg |
| Dibasic calcium phosphate anhydrous | 386.82 | mg |
| | 800.00 | mg |

Formula 2 - with RYR standard:

| | | |
|---|---|---|
| Microcrystalline cellulose | 200.92 | mg |
| Magnesium stearate | 8.00 | mg |
| Silicum dioxide | 4.00 | mg |
| Folic Acid | 0.26 | mg |
| Standard Red Yeast Rice (1.75%) | 200 | mg |
| Dibasic calcium phosphate anhydrous | 386.82 | mg |
| | 800.00 | mg |

Both formulations have been stored in the same blister, composed of PVC/PVDC (250/40 microns) in the front blister, and aluminium in the retroblister. The storage conditions were 40° C. and 75% relative humidity (RH) for 6 months (a standard accelerated stability test).

The monacolin K contents has been analysed after 0, 1, 3 and 6 months with HPLC-DAD.

The results are shown in table 2 and FIG. 2. The formulation comprising the encapsulated red yeast rice showed a significantly higher stability compared to the not encapsulated red yeast rice after all time points.

TABLE 2
% Reduction of monacolin K at different time points

Reduction of monacolin K
after incubation in months

| | 0 | 1 | 3 | 6 |
|---|---|---|---|---|
| Formula 1 | 0% | −1% | −8% | −10% |
| Formula 2 | 0% | −13% | −27% | −40% |

### Preparation of Granulates and Particle Size Distribution Analysis

Three granulates (a)-(c) were prepared according to the process set out in the description:
heating one or more of the lipid carrier components until at least partially melted, preferably completely melted;
combining the red yeast rice with the one or more melted lipid carrier components until the red yeast rice is incorporated into the molten lipid carrier matrix;
combining the resulting mixture with any remaining lipid carrier components;
cooling the resulting mixture until the lipid carrier components have solidified; and
crushing, and optionally sieving, the resulting solid mixture to obtain a granulate.

In preparing these granulates, the melting phase temperature, and the mixing speed (for massing) were varied slightly:

| | | | | Particle size |
|---|---|---|---|---|
| | | Mixing | Mixing | distribution |
| | Melting phase | speed | speed | (Dv(10), |
| | temperature - | (melting) - | (massing) - | Dv(50), |
| Granulate | ° C. | rpm | rpm | Dv(90)) - μM |
| (a) | 65 | 60 | 60 | Dv(10) - 16.6 |
| | | | | Dv(50) - 58.7 |
| | | | | Dv(90) - 153.3 |
| (b) | 68 | 60 | 90 | Dv(10) - 21.3 |
| | | | | Dv(50) - 72.1 |
| | | | | Dv(90) - 512.6 |
| (c) | 68 | 60 | 60 | Dv(10) - 19.2 |
| | | | | Dv(50) - 66.3 |
| | | | | Dv(90) - 351.6 |

Size distribution measurements were performed by laser light scattering (LLS) according to ISO 13320:2020 using a Mastersizer 3000 (Malvern-Panalytical) and samples were analysed as such (dry powder).

Particle size parameters (Dv(10), Dv(50), Dv(90)) are expressed in terms of the equivalent spherical diameter in volume. The equivalent spherical diameter is the diameter obtained from the laser diffraction analyses. It is the diameter of a sphere with a volume equivalent to that of the analysed particle.

In preparing the granulates it was found that each of the three granulates (a)-(c) had acceptable chemical and physical characteristics.

From a manufacturing point of view, granulate (b) was optimal. This is because the particle size distribution of this granulate improved flowability (compared to granulates (a) and (c)) which in turn improved case of handling of the granulate during the subsequent formulation steps (mixing and eventually tableting).

1. A composition comprising red yeast rice and a lipid carrier comprising (a) a fatty alcohol and/or a fatty acid; (b) a glyceride; and (c) a polyethylene glycol.

2. The composition of claim 1, wherein the lipid carrier encapsulates the red yeast rice or is intimately mixed with the red yeast rice.

3. The composition of claim 1, wherein the red yeast rice is standardized in monacolin K to 1.75% w/w.

4. The composition of claim 1, wherein the fatty alcohol is selected from the group consisting of cetyl alcohol, stearyl alcohol, palmityl alcohol, myristyl alcohol, arachidyl alcohol, lauryl alcohol, behenyl alcohols, and combinations thereof.

5. The composition of claim 1, wherein the fatty acid is selected from the group consisting of stearic acid, palmitic acid, myristic acid, arachidic acid, lauric acid, and combinations thereof.

6. The composition of claim 1, wherein the glyceride is selected from the group consisting of glyceryl monostearate, glyceryl distearate, glyceryl behenate, glyceryl dibehenate, glyceryl tristearate, glyceryl laurate, glyceryl palmitate, glyceryl myristate, glyceryl arachidate, and combinations thereof.

7. The composition of claim 1, wherein the polyethylene glycol has an average molecular mass of from 400 to 6000 g/mol.

8. The composition claim 1, wherein the lipid carrier comprises cetyl alcohol, glyceryl monostearate, glyceryl dibehenate, and PEG 1500.

9. The composition of claim 1, wherein the composition is obtainable by hot melt granulation.

10. The composition of claim 1 for use in the treatment or prevention of hypercholesterolemia or hyperlipidemia.

11. The composition of claim 1, additionally comprising one or more components selected from the group consisting of Berbens aristata, phytosterols and/or phytostanols, Cynara cardunculus extract, Citrus bergamia extract, Allium sativum, Salvia miltiorrhiza, policosanol, Camellia sinensis extract, Melannurca campana extract, Curcuma longa and curcuminoids, spirulina, chitosan, betaglucan, and glucomannan.

12. The composition claim 1, additionally comprising one or more components selected from the group consisting of coenzyme Q10, astaxanthin, folic acid, and orthosiphon.

13. A process for preparing a composition of claim 1, comprising the following steps:
(i) heating one or more of the lipid carrier components until at least partially melted;
(ii) combining the red yeast rice with the one or more melted lipid carrier components until the red yeast rice is incorporated into a molten lipid carrier matrix;
(iii) combining the resulting mixture with any remaining lipid carrier components;
(iv) cooling the resulting mixture until the lipid carrier components have solidified; and
(v) crushing, and optionally sieving, the resulting solid mixture to obtain a granulate.

14. The process of claim 13, wherein in step (i), all of the lipid carrier components are heated until they are completely melted.

15. The process of claim 13, wherein the red yeast rice is combined with the melted lipid carrier components gradually in portions, until the red yeast rice is completely incorporated into the molten lipid carrier matrix.

16. The composition of claim 7, wherein the polyethylene glycol has an average molecular mass selected from the group consisting of 400 g/mol, 1500 g/mol, 3350 g/mol, 4000 g/mol, and 6000 g/mol.
